(19) United States
(12) Patent Application Publication
(10) Pub. No.: US 2015/0320766 A1
(43) Pub. Date: Nov. 12, 2015

(54) COPPER INTRAUTERINE DEVICE

(71) Applicants: LABORATOIRE HRA-PHARMA, Paris (FR); THE POPULATION COUNCIL, INC., New York, NY (US)

(72) Inventors: André ULMANN, PARIS (FR); Erin GAINER, PARIS (FR); Delphine LEVY, BAGNOLET (FR); Christine SEGUIN, PARIS (FR); Florian BATTUNG, PARIS (FR); Regine SITRUK-WARE, NEW YORK, NY (US)

(21) Appl. No.: 14/651,812

(22) PCT Filed: Dec. 13, 2013
(86) PCT No.: PCT/EP2013/076471

§ 371 Date: Jun. 12, 2015

Related U.S. Application Data
(60) Provisional application No. 61/738,056, filed on Dec. 17, 2012.

(30) Foreign Application Priority Data
Dec. 14, 2012 (EP) 12306591.4

(51) Int. Cl. A61K 31/57; A61K 33/34; A61K 47/34; A61K 9/00

(52) U.S. Cl. CPC A61K 31/57; A61K 9/0039; A61K 33/34; A61K 47/34

(57) ABSTRACT

The invention relates to a copper contraceptive intrauterine system (IUS) with a flexible frame, which system is further capable of releasing a selective progesterone receptor modulator (SPRM), such as ulipristal acetate, for reducing or preventing bleeding side effects.

The present invention relates to an improved intrauterine system for reversible contraceptive effect over a prolonged period of time in a female mammal. More particularly, the invention relates to an intrauterine system with reduced bleeding side effects.

### BACKGROUND OF THE INVENTION

Intrauterine devices (IUD) represent the most cost-effective reversible method of contraception to date, due to their high effectiveness and long duration of action. A large diversity of IUD has been developed. Two main types of IUD exist, presented as non-hormonal IUD and hormonal IUD (also called intra-uterine system or IUS) respectively.

The most classic and oldest non-hormonal IUD consists of a plastic frame surrounded with a copper wire. The copper IUD acts as a spermicide that inhibits sperm motility and viability within the uterus. Spermatozoids travel through the cervical mucus is restricted and the majority of spermatozoids are destroyed within the endometrium and fallopian tubes. One of the most advantageous effects is that copper IUD works immediately upon insertion and fertility returns quickly upon removal. In addition, the lifespan of a copper IUD ranges from about 2 to 12 years and thereby is a very long-acting lasting method of birth control and one of the most effective method of contraception with no compliance issues.

For 20 years, a second generation of IUD has been developed, called hormonal IUD or IUS, which acts differently from copper IUD. The hormonal IUD has a plastic frame containing and releasing a progestin, most commonly levonorgestrel (LNG). A small amount of hormone is continuously released into the uterus, thickening cervical mucus and blocking spermatozoids movement which then cannot enter the cervix and reach the egg. This is a additional mechanism of action preventing fertilization. The uterine lining thickness is also decreased, making it inhospitable for fertilized eggs to implant. In addition, the IUS brings an additional benefit of significantly reducing menstrual bleeding, and finally in a small proportion of women ovulation is inhibited. Current hormonal IUD may be effective up to 5 years

However, even if women appreciate IUD's ease of use and permanent effect over a long period of time in absolute term, related adverse effects lead to discontinuation in many women. Indeed, for the first six months after insertion, both hormonal and copper IUD may cause irregular spotting and bleedings. In addition, copper IUD is often responsible for an increased amount of menstrual blood flow during periods. Finally, the IUS may cause hormonal adverse events including a slight increase of vaginal dryness, hot flushes, headaches, nausea, acne and hirsutism.

Thus, a need exists for an improved IUD sufficiently efficient for allowing an effective birth control for several years and that does not cause latent and/or unacceptable side effects.

### SUMMARY OF THE INVENTION

The inventors now propose a new copper IUD, with the properties of a non-hormonal IUD, while inducing a reduced risk of excessive bleedings.

More particularly, the inventors propose a copper IUD designed to release a selective progesterone receptor modulator (SPRM), such as ulipristal acetate (UPA). The improved intrauterine system thus uses copper as the contraceptive agent for prolonged birth control and a SPRM as a bleeding control agent. The amount of SPRM released within the intrauterine cavity during the IUS implantation is preferably maintained below the antiovulatory contraceptive level.

Then, it is an object of the invention to provide a copper intrauterine device with a flexible frame, which is capable of releasing a selective progesterone receptor modulator (SPRM) at a controlled continuous rate.

The intrauterine system (IUS) of the invention combines copper's contraceptive effect and SPRM bleeding control effect.

In an embodiment, the IUS comprises a polymeric matrix containing the SPRM, which is permeable to said SPRM. The polymeric matrix advantageously allows a controlled release of the SPRM within a uterine cavity. The IUS can be formed partly or entirely with said polymeric matrix containing the SPRM. In some embodiments, the polymeric matrix containing the SPRM is coated with a rate-controlling polymeric membrane.

The invention further provides a kit comprising a copper IUS as defined above and an inserter for inserting and positioning said copper IUS releasing SPRM.

The invention further provides a method for providing contraception in a female mammal, comprising the steps consisting in:
a) Providing the copper IUS as defined above; and
b) Inserting said intrauterine system into the uterine cavity of said female mammal.

Advantageously, the method in addition to providing contraception further reduces or prevents bleedings in the female.

According to the method of the invention, the IUS may be maintained in position up to 10 years, preferably up to 7 years, more preferably up to 5 years, and even more preferably up to 3 years.

The invention still further provides a SPRM for use in reducing or preventing bleeding side effects in a female mammal who carries a copper intrauterine system, wherein the copper intrauterine system is designed to release the SPRM.

The SPRM acts as a reducing bleeding agent that may stop or reduce spotting and bleeding due to the copper presence in the uterine cavity.

At last, the invention provides a SPRM for use in treating a gynecological disease in a female mammal, wherein the SPRM is released by a copper intrauterine device in the uterine cavity of the female mammal.

### BRIEF DESCRIPTION OF THE FIGURES

The invention is further illustrated by the following figures, describing particular constructions of the IUS according to the invention.

FIG. 1 illustrates an IUS of the invention having a T-shaped frame made with a polymeric matrix containing the SPRM to diffuse progressively and continuously within a uterine cavity. A copper wire, and more particularly 3 segments thereof, is coiled around the two arms and the stem of the T shape.

FIG. 2 illustrates an IUS of the invention having a T-shaped frame made with a polymeric matrix. A reservoir is connected to the stem of the T shape. The reservoir is made with a polymeric matrix containing the SPRM and suitable to release said SPRM at a controlled rate. Copper sleeves are mounted on the arms of the T-shape.

FIG. 3 illustrates an IUS of the invention having a T-shaped frame made with a polymeric matrix. A reservoir is connected to the stem of the T shape. The reservoir is made with a polymeric matrix containing the SPRM, which is wrapped in a rate-controlling polymeric membrane suitable to provide an appropriate daily release of the SPRM in the uterine cavity. A copper wire is coiled around each arm of the T-shape.

### DETAILED DESCRIPTION OF THE INVENTION

The inventors propose to use a SPRM as bleeding control agent in association with a copper IUD allowing birth control while avoiding bleedings by a direct effect on the endometrium.

In the context of the invention, a “copper IUD”, “copper IUS”, “copper-bearing IUD” or “copper-bearing IUS” refers to an IUD or IUS wherein the contraceptive effect is at least provided by copper. The copper IUD or IUS may comprise any suitable forms of copper, such as copper wire or sleeve, copper coating, powdered copper mixed in the frame of the IUD or IUS, etc.

### Selective Progesterone Receptor Modulator

The invention relates, generally, to an improved copper intrauterine system releasing a SPRM in the uterine cavity wherein the IUS is inserted to reduce or prevent bleeding side effects.

In the context of the invention, a SPRM is a compound that is a progesterone analog and that has a mixed agonist/antagonist profile of action, which is tissue specific. A SPRM may act as an agonist in some tissues while as an antagonist in others. One skilled in the art may easily determine if a ligand is a SPRM or not, for example by reference to the articles of Smith and O'Malley, Endocrine Review, 25(1):45-71, and/or of Chabbert-Buffet et al., Human Reproduction Update, 2005, 11, 293-307.

The SPRM may be a non-steroidal compound or steroidal derivative. Examples of non-steroidal SPRM are cited in: Dong et al., Steroids, 2004, 69:201-207, Zhi et al., J Med Chem, 2003, 46:4104-4112 and Zhi et al., Curr Top Med Chem, 2008, 8:766-780.

Preferably, steroidal derivative SPRM are chosen among steroidal derivatives with a substituted aryl group in position 11β. Preferred aryl groups comprise the 4-(dimethylamino)phenyl, the 4-acetylphenyl and the benzaldoxime as well as 4-methylamino-phenyl and 4-aminophenyl. Examples of such steroidal SPRM may be found in the following publications: Rao et al., Steroids, 1998, 63:523-530 and Chabbert-Buffet et al., Human Reproduction Update, 2005, 11, 293-307. More particularly, Chabbert-Buffet et al. discloses mifepristone, onapristone, asoprisnil, ulipristal acetate (UPA) or active metabolites thereof, Org 33628 and Org 31710 as SPRM. A further example of SPRM is telapristone (CDB-4124) and metabolites thereof.

In a preferred embodiment, the selective progesterone receptor modulator is ulipristal acetate. Ulipristal acetate, formerly known as CDB-2914, is 17α-acetoxy-11β-(4-N,N-dimethylamino-phenyl)-19-norpregna-4,9-diene-3,20-dione, represented by formula I:

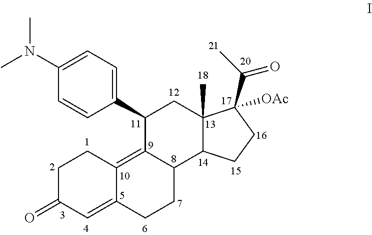 [SMILES: CC(=O)O[C@]1(C(C)=O)CCC2C3CCC4=CC(=O)CCC4=C3[C@@H](c3ccc(N(C)C)cc3)C[C@@]21C]

It is a well-known steroid, more specifically a 19-norprogesterone, which possesses antiprogestational and antiglucocorticoidal activity. This compound, and methods for its preparation, are described in U.S. Pat. Nos. 4,954,490, 5,073,548, and 5,929,262, and international patent applications WO2004/065405 and WO2004/078709. Properties of this compound are further described in Blithe et al, 2003, Steroids, 68:1013-1017 and Gainer and Ulmann, 2003, Steroids, 68:1005-1011.

Metabolites of CDB-2914, include those described in Attardi et al, Journal of Steroid (CDB-3877); didemethylated CDB-2914 (CDB-3963); 17alpha-hydroxy CDB-2914 (CDB-3236); aromatic A-ring derivative of CDB-2914 (CDB-4183).

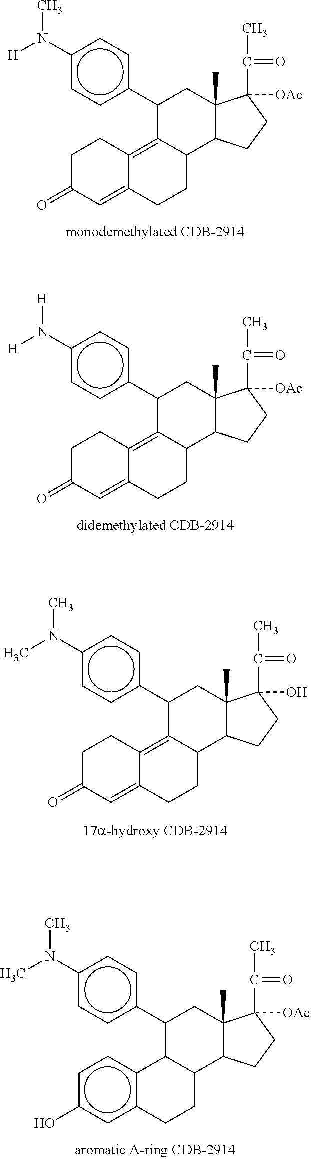

The copper IUS of the invention is able to release an effective amount of SPRM within the uterine cavity of a female mammal, so that bleedings generally connected to the presence of a copper intrauterine device are reduced or stopped. According to the invention, the purpose of the SPRM is to minimize potential blood loss caused by the copper presence in the uterine cavity by a direct effect on the endometrium without systemic effect, while the birth control action is provided by copper. The amount of SPRM which daily diffuses within the uterine cavity is thus sufficient to reduce or suppress bleedings associated with the use of a copper IUD. In a preferred embodiment, the daily dose of SPRM diffusing within the uterine cavity is insufficient to interfere with, or block, ovulation. Preferably the daily dose is insufficient to provide a contraceptive action itself.

Typical dose ranges of SPRM released in the uterine cavity, suitable and sufficient for reducing bleedings caused by the copper presence are from 5 μg to 100 μg, e.g. from 20 μg to 100 μg per day. That way, the serum level of SPRM is maintained below 1 ng/ml, which is insufficient to block ovulation. A daily dose from 5 μg to 100 μg encompasses a daily dose from 5 μg to 10 μg, from 10 μg to 20 μg, from 20 μg to 30 μg, from 30 μg to 40 μg, from 40 μg to 50 μg, from 50 μg to 60 μg, from 70 μg to 80 μg, from 90 μg to 100 μg.

In a particular embodiment, the copper IUS of the invention is able to daily diffuse between 5 μg to 100 μg, e.g. between 20 μg and 100 μg, preferably between 40 μg and 80 μg, and more preferably 60 μg of SPRM. Preferably, the copper IUS releases a substantially constant low amount of SPRM over a prolonged period of time.

The intrauterine system of the invention may advantageously contain sufficient amounts of SPRM to bring the desired effect over a prolonged period of time, preferably at least 1 year, and more preferably up to at least 3 years. For example, the IUS comprises between 10 mg and 400 mg of SPRM in total, preferably between 50 mg to 400 mg of SPRM, more preferably between 100 mg and 200 mg, even more preferably 150 mg, so that it may release a substantially constant daily amount of SPRM comprised between 5 μg and 100 μg, preferably from 20 μg to 100 μg over a period of time comprised between 3 and 5 years.

In a particular embodiment, the copper IUS comprises two or more SPRM, contained in a same or in different polymeric matrix, each forming at least part of the IUS frame. In such a case, the amounts disclosed above correspond to the total amount of all the SPRM.

In a most preferred embodiment, the IUS of the invention does not comprise any other progesterone or oestrogen analogues.

### Polymeric Matrix

The SPRM is contained in a polymeric matrix which is permeable to the passage of said SPRM so that the SPRM may diffuse within the uterine cavity. More particularly, the SPRM is mixed in the polymeric matrix, and gradually diffuses through said polymeric matrix within the intrauterine cavity. Preferably, the SPRM is substantially homogeneously dispersed throughout the polymeric matrix.

Advantageously, the polymer allows a high solubility of the SPRM in the corresponding polymeric matrix. Preferably, the polymers of the polymeric matrix may not be absorbed in the vaginal tract or in the uterine cavity of the female using the corresponding IUS. In addition, the polymer matrix does not induce an excessive or contra-indicated tissue reaction at the site of placement of the IUS in the female uterus.

The SPRM-permeable polymeric matrix may be formed with any thermoplastic polymer or elastomer suitable for pharmaceutical use. For instance, the polymer may be a silicone rubber or a silicone elastomer, in particular a polyorganosiloxane such as polysiloxanes, polydimethylsiloxane, copolymers of dimethylsiloxane and methylvinylsiloxane and derivatives thereof. In particular, suitable polymers include, without limitation, polysiloxanes, polydimethylsiloxanes, polyurethane, polyethylene, ethylene-vinyl acetate copolymers (EVA), cellulose, polyacrylates, etc. In some embodiments, SPRM-permeable polymeric matrix is made of a polymer selected from ethylene-vinyl acetate copolymers (EVA), polyorganosiloxanes and combinations thereof.

In some embodiments, the SPRM-permeable polymeric matrix is reticulated. The reticulation of the matrix may be obtained by vulcanization or by chemical curing in the presence of a chemical cross-linking agent and/or a curing catalyst.

The polymers and their physical properties are known in the art as well as their process of synthesis (see also Encyclopedia of Polymer Science and Technology—Interscience Publishers, Inc., NY, 1971; and Handbook of Common Polymers, Scott and Roff—CRC Press, Cleveland, Ohio, 1971). Since the rate of passage of a compound through a polymer is dependent on the molecular weight and solubility of the compound therein, one skilled in the art may vary the composition and properties of the polymeric matrix to adapt and control the dosage rate per area of the IUS. In addition, the release of the SPRM may be also controlled by modifying/adapting the surface area of the polymeric matrix containing said SPRM.

In a particular embodiment, the polymeric matrix may be coated, or surrounded, with a coating suitable for regulating SPRM release. For example, the polymeric matrix containing the SPRM may be encased in a rate-controlling polymeric membrane (also called herein release-controlling polymeric membrane). Said rate-controlling polymeric membrane may be appropriate to control the daily release of SPRM from the polymeric matrix and thus may be permeable to the SPRM. Furthermore, the rate-controlling polymeric membrane may be particularly useful to avoid or at least reduce initial burst of SPRM subsequent to the insertion of the IUS within the uterine cavity. The rate-controlling polymeric membrane may be made of similar or distinct polymers as compared to the polymeric matrix containing the SPRM. For instance, the rate-controlling polymeric membrane is made of a polyorganosiloxane such as polydimethylsiloxane or other polymers such as ethylene-vinyl acetate.

The rate-controlling polymeric membrane or layer may have a thickness ranging from 0.1 mm to 1 mm such as 0.5 mm.

In one embodiment, the SPRM is mixed in the form of a powder with the polymeric material, to form a polymeric mixture, which may be then molded, extruded, and/or casted so as to obtain an appropriate shape. Optionally, the polymeric mixture may be vulcanized or chemically cured so as to obtain an appropriate reticulation. Otherwise, the SPRM may be dissolved in an appropriate solvent, such as dichloromethane, to form a solution which is mixed with the polymeric material, to form the polymeric mixture. Alternatively, both the polymer and the SPRM in the form of powders can be mixed and then molded under adequate conditions, through injection, extrusion etc.

The weight ratio of the SPRM to the polymeric material forming the SPRM-permeable polymeric matrix is typically from 0.01 to 2, preferably from 0.1 to 1.

According to the invention, the polymeric matrix containing the SPRM forms at least part of the frame of the copper IUS. For example, the polymeric matrix containing the SPRM may form the core or main body of the frame, or only a segment of said frame. In a particular embodiment, the polymeric matrix containing the SPRM forms a segment of the frame that is fixed to the main body of the device. In another particular embodiment, the polymeric matrix containing the SPRM forms an additional reservoir locked with the main body by any suitable means. Various sizes and shapes may be manufactured. For example, the reservoir has a ring or tubular shape, which surrounds the outer surface of the frame. Otherwise, the reservoir may be fixed to a portion or extremity of the frame, or may be incorporated within the frame, etc. In a still further embodiment, the frame of the IUS is entirely made of the polymeric matrix containing the SPRM, which may be optionally coated with a rate-controlling polymeric membrane.

When the polymeric matrix containing the SPRM forms only part of the frame, the main body of said frame is made of a polymer or mixture of polymer, which may be same or different from the polymeric matrix containing the SPRM. Preferably, the polymeric matrix containing the SPRM is made with a softener polymer than the frame. For example, the frame is made with polyurethane or polyethylene, and the SPRM reservoir is made with ethylene-vinyl acetate (EVA) or polydimethylsiloxane. In a preferred embodiment, the frame is made with polyethylene, and the polymeric matrix containing the SPRM is made of EVA wherein micronized UPA is dispersed.

### Copper's Contraceptive Effect

According to the invention, the active contraceptive substance of the IUS is copper. Copper may be in any forms suitable to load the IUS. The copper release is preferably constant over a long period of time and preferably up to at least 3 years. All the models of copper IUD of the art may be used for manufacturing the copper IUS of the invention.

Advantageously, the surface area of the copper is comprised between 200 and 400 mm², preferably between 250 and 380 mm².

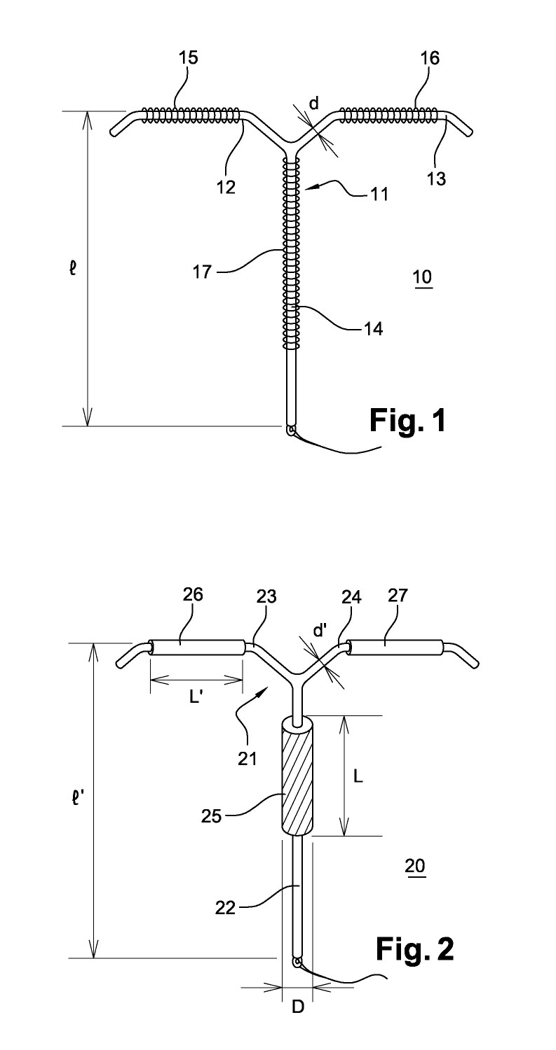

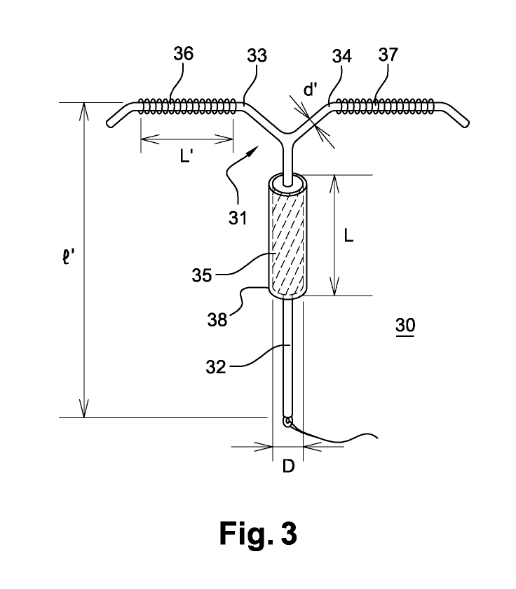

For example, in a particular embodiment, the IUS comprises at least one copper wire, which interacts with at least part of the frame of the IUS. For example, as shown in FIG. 1 or FIG. 3, the copper wire surrounds part of the outer surface of the T-shaped frame. In another example the copper wire may be fixed along a portion of the frame or incorporated within the frame.

In another embodiment, the IUS comprises one or several sleeve(s) fixed around the outer surface of the plastic frame of the IUS. For example, as shown in FIG. 2, the IUS comprises two copper sleeves, each partially surrounding the outer surface of one or two arms of the T-shaped frame.

Otherwise, or in addition, the IUS may be at least partially coated by a copper coating fixed on the plastic frame of the IUS by means of a thin diffusion layer.

In another embodiment, the IUS comprises a polymeric matrix containing powdered copper distributed throughout. The polymeric matrix containing the copper powder may form the core or main body of the frame, as well as only a segment of said frame. Preferably, the frame of the IUS is entirely made of the polymeric matrix containing powdered copper. In a particular embodiment, the polymeric matrix containing powdered copper further contains the SPRM. In another embodiment, the SPRM and the powdered copper are contained in different part of the shape of the IUS, made of same or different polymeric matrix.

### IUS General Shape

According to the invention, the IUS may be manufactured in any shape and size designed and adapted for placing in the female uterine cavity and lying in the required position for a long term insertion, for example from one to several years, preferably from 1 to 12 years, more preferably from 2 to 5 years and even more preferably up to at least 3 years.

Preferably, the copper IUS is sufficiently resilient and flexible to allow adaptation to various sizes and shapes of the uterus, and to avoid irritation of the endometrium. In the context of the invention, a “flexible frame” means that the frame of the IUS can be deformed easily by applying a pressure and can return to its original shape upon relieving of the pressure. Such flexibility is useful for inserting, using and removing the IUS.

For example, the IUS may have a T-shaped, Y-shaped, C-shaped, O-shaped or Omega-shaped frame. The size of the frame will depend on the average sized uterine cavity of the female, and is advantageously able to avoid movement and/or rotation inside said uterine cavity. For human female, the lengthways dimension of the frame is typically from 10 to 40 mm, preferably from 20 to 35 mm. The cross sectional diameter of the segments is typically from 0.5 to 10 mm, preferably from 1 to 5 mm.

In some embodiments, the IUS of the invention comprises at least one copper wire, which interacts with at least part of the frame of the IUS, and at least one reservoir made of a SPRM-permeable polymeric matrix which contains SPRM and which may be optionally coated with a rate-controlling polymeric membrane.

Particular embodiments of IUS shapes of the invention are further illustrated by the following non limitative examples of IUS dedicated to women.

The intrauterine system 10 illustrated FIG. 1 has a T-shaped frame 11 completely made with a same polymeric material, such as polyethylene or polyacrylate matrix. The SPRM, such as UPA, is dispersed homogeneously throughout this matrix. For example, 200 mg of UPA (CDB-2914) are dissolved in 2 mL of ethylacetate. Next, 1800 mg of polyethylene are mixed with the solution of UPA. The mixture is molded to form a T-shaped frame with a lengthways dimension 1 of 30 mm, and a cross sectional diameter d of the rods of 1 mm and solvent is evaporated. Three segments 15, 16, 17 of copper wire are coiled respectively around the two arms 12 and 13 and the stem 14 of the T, so that the copper area of the IUS 10 is about 380 mm².

The intrauterine system 20 shown in FIG. 2 has a T-shaped frame 21 bearing a reservoir 25 containing the SPRM. For example, the polymeric matrix containing the SPRM is an ethylene-vinyl acetate (EVA) matrix comprising 28% by weight of vinylacetate, while the polymeric matrix of the frame is a polyacrylate matrix. The reservoir 25 may be obtained by mixing 150 mg of powdered UPA (CDB-2914) with 150 mg of the matrix powder. The matrix/UPA powders are dissolved in organic solvent, extruded into a mold and solvent is evaporated, creating a 300 mg hollow tube, with a lengthways dimension L of 20 mm, an outer diameter D of 4 mm and a cross section of 1.6 mm. The T-shaped frame 21 may be obtained by molding 2000 mg of polyacrylate, to obtain T-shaped frame with a lengthways dimension 1′ of 35 mm, and a cross sectional diameter d′ of the rods of 0.9 mm. The tubular reservoir 25 is mounted and fixed around the stem 22 of the T. The IUS further comprises two copper sleeves 26 and 27, each being coiled round an arm 23, 24 of the T. The lengthways dimension L′ of each sleeve 26, 27 is 10 mm, so that the copper area of the IUS 20 is about 380 mm2.

The intrauterine system 30 shown in FIG. 3 has a T-shaped frame 31 bearing a reservoir 35 containing the SPRM. For example, the polymeric matrix containing the SPRM is made of ethylene-vinyl acetate (EVA) matrix or a polydimethylsiloxane matrix, while the polymeric matrix of the frame is made of a polyacrylate matrix. The reservoir 35 may contain 0.8 mg of ulipristal acetate per mg of polymeric matrix. The reservoir 35 may be obtained as described above for FIG. 2 and may have the form of a hollow tube, with a lengthways dimension L of 20 mm, an outer diameter D of 4 mm and a cross section of 1.6 mm. The T-shaped frame 31 may be obtained by molding 2000 mg of polyacrylate, to obtain T-shaped frame with a lengthways dimension 1′ of 35 mm, and a cross sectional diameter d′ of the rods of 0.9 mm. The tubular reservoir 35 is mounted and fixed around the stem 32 of the T. The tubular reservoir 35 is coated with a rate-controlling polymeric membrane 38. The rate-controlling polymeric membrane 38 may be made of silicone elastomer such as polydimethylsiloxane. The thickness of membrane 38 is controlled when moulding to 0.5 mm. The rate-controlling polymeric membrane 38 may be inserted around the reservoir and cut to the appropriate dimension, or may be molded around the tubular reservoir 35. Two segments of copper wires 36 and 37 are coiled respectively around the two arms 33 and 34 of the T so that the copper area of the IUS 30 is about 380 mm².

According to the invention, the copper IUS may be inserted and positioned into the uterus of the female mammal by means of a separate inserter. The copper IUS may be packaged and used together with such an inserter. The copper IUS and the inserter may be sterilized, e.g. by exposure to gamma radiation or to ethylene oxide. The sterilization may be performed after packaging.

Advantageously, the inserter allows introduction of the IUS in a compressed state in order to facilitate the insertion of said IUS into the cervical canal.

Accordingly, the invention further provides a kit comprising a copper IUS as described above and a separate inserter suitable for inserting and positioning said copper IUS within the uterus of a female mammal.

For example, the inserter comprises a tube in which the IUS can be housed prior to insertion and means for removing the IUS. Preferably, the IUS is slidably mounted into the tube, so that a plunger of the inserter can push said IUS outside the tube when the IUS is correctly positioned. The inserter may further comprise stopping means facilitating correct position of the IUS within the uterus.

### Contraceptive Methods

The intrauterine system of the invention may be used as a safe and effective contraceptive for preventing pregnancy in any female mammal of child-bearing age, and more particularly in any women of child-bearing age. The IUS of the invention may stay within the uterine cavity of a female mammal over a prolonged period of time, and advantageously up to at least 3 years.

Accordingly, the invention provides a method for preventing pregnancy in a female mammal comprising the steps consisting in:
a) Providing the copper intrauterine system as defined above; and
b) Inserting said intrauterine system into the uterine cavity of said mammal.

Advantageously, the method of the invention further reduces or prevents bleedings connected to the presence of the copper in the female mammal.

Thus, the method of the invention proposes to combine copper's contraceptive effect and SPRM reducing bleeding side effect. All embodiments of copper IUS disclosed above may be used for implementing said method and preventing pregnancy in a female mammal. The IUS of the invention acts immediately upon insertion, as contraceptive by releasing an effective contraceptive amount of copper ions and as reducing bleeding device by releasing an effective amount of SPRM, such as UPA.

The present invention further discloses a SPRM for use in preventing bleeding side effects in a female mammal who carries a copper intrauterine device, wherein the copper intrauterine device is designed to release the SPRM in the uterine cavity of the female mammal. Advantageously, the SPRM is to be administered with a daily non contraceptive dosage, preferably comprised between 5 μg and 100 μg, e.g. between 20 μg to 100 μg, more preferably comprised between 40 μg and 80 μg of SPRM, and even more preferably equal to 60 μg.

1. A copper intrauterine system (IUS) with a flexible frame, which system is further capable of releasing a selective progesterone receptor modulator (SPRM).

2. The copper intrauterine system of claim 1, wherein the SPRM is ulipristal acetate (UPA) or a metabolite thereof.

3. The copper intrauterine system of claim 1, wherein the SPRM is contained in a polymeric matrix permeable to the passage of said SPRM.

4. The copper intrauterine system of claim 3, wherein the polymeric matrix is a thermoplastic polymer selected from the group consisting of polyorganosiloxanes polyurethane, polyethylene, ethylene-vinyl acetate copolymers, cellulose, polyacrylates and polyamides.

5. The copper intrauterine system of claim 3, wherein the SPRM is dispersed throughout the polymeric matrix.

6. The copper intrauterine system of claim 3 wherein the polymeric matrix containing the SPRM is coated with a rate-controlling polymeric membrane.

7. The copper intrauterine system of claim 1, suitable for daily diffusing a non-contraceptive amount of SPRM within the uterine cavity.

8. The copper intrauterine system of claim 7, suitable for daily diffusing between 5 μg and 100 μg of SPRM within the uterine cavity.

9. The copper intrauterine system of claim 1, which comprises between 10 mg and 400 mg of SPRM in total.

10. The copper intrauterine system of claim 3, wherein the polymeric matrix containing the SPRM (i) forms at least part of the frame, or (ii) is connected to the frame.

11. The copper intrauterine system of claim 10, wherein the polymeric matrix containing the SPRM is connected to the frame and the frame is made of polymer or mixture of polymers which is same or different from the polymeric matrix containing the SPRM.

12. The copper intrauterine system of claim 1, comprising a copper wire interacting with at least part of the frame.

13. The copper intrauterine system of claim 1, wherein the intrauterine system has a T-shaped, Y-shaped, C-shaped, O-shaped or Omega-shaped frame.

14. A method for providing contraception in a female subject comprising
a) Providing an copper intrauterine system as defined in claim 1; and
b) Inserting said intrauterine system into the uterine cavity of said subject.

15. A method for providing contraception to a female subject, said method comprising the step of inserting a copper intrauterine device in the uterine cavity of the female subject, wherein the copper intrauterine device releases a selective progesterone receptor modulator (SPRM) in the uterine cavity of the female subject.

16. A method for treating a gynecological disease in a female subject, said method comprising the step of inserting the copper intrauterine device of claim 1 in the uterine cavity of the female subject.

17. The copper intrauterine system of claim 2, wherein the metabolite of ulipristal acetate is CDB-3877 or CDB-3963.

18. The copper intrauterine system of claim 3, wherein the polymeric matrix is a thermoplastic polymer selected from the group consisting of polysiloxane and polydimethylsiloxane.
